(19) United States
(12) Patent Application Publication
(10) Pub. No.: US 2023/0390324 A1
(43) Pub. Date: Dec. 7, 2023

(54) TREATMENT OF FATTY LIVER DISEASES

(71) Applicant: TX MEDIC AB, Viken (SE)

(72) Inventors: Lars BRUCE, Viken (SE); Adam BRUCE, Viken (SE)

(21) Appl. No.: 18/246,570

(22) PCT Filed: Sep. 17, 2021
(86) PCT No.: PCT/SE2021/050895

§ 371 Date: Mar. 24, 2023

(30) Foreign Application Priority Data
Sep. 29, 2020 (SE) 2051132.5

(51) Int. Cl. A61K 31/737; A61P 1/16

(52) U.S. Cl. CPC A61K 31/737; A61P 1/16

(57) ABSTRACT

The invention relates to dextran sulfate, or a pharmaceutically acceptable salt thereof, for use in improving liver function in a subject suffering from a non-alcoholic fatty liver disease (NAFLD). The dextran sulfate, or the pharmaceutically acceptable salt thereof, has an average molecular weight equal to or below 10 000 Da and is formulated for intravenous or subcutaneous administration to the subject.

### TECHNICAL FIELD

The present invention generally relates to fatty liver diseases, and treatment thereof using dextran sulfate, or a pharmaceutically acceptable salt thereof.

### BACKGROUND

Fatty liver disease (FLD), also known as hepatic steatosis, is a condition where excess fat builds up in the liver. There are two types of fatty liver diseases: non-alcoholic fatty liver disease (NAFLD) and alcoholic liver disease (ALD).

NAFLD, also known as metabolic (dysfunction) associated fatty liver disease (MAFLD), is excessive fat build-up in the liver. NAFLD is the most common liver disorder worldwide and is present in approximately 25% of the world's population. NAFLD is the leading cause of chronic liver disease and the second most common reason for liver transplantation in the U.S. and Europe as of 2017. Obesity and type 2 diabetes are strong risk factors for NAFLD. Other risks include being overweight, metabolic syndrome, a diet high in fructose, and older age.

Current treatments for NAFLD include weight loss by dietary changes and exercise. There is tentative evidence for pioglitazone and vitamin E having a positive effect on NAFLD.

ALD, also called alcohol-related liver disease (ARLD), is a term that encompasses the liver manifestations of alcohol overconsumption, including fatty liver, alcoholic hepatitis, and chronic hepatitis with liver fibrosis or cirrhosis. It is the major cause of liver disease in Western countries. More than 90% of all heavy drinkers develop fatty liver whilst about 25% develop the more severe alcoholic hepatitis, and 15% cirrhosis.

Corticosteroids are sometimes used for treatment of ALD. However, this is recommended only when severe liver inflammation is present. Silymarin has been investigated as a possible treatment, with ambiguous results. The effects of anti-tumor necrosis factor medications, such as infliximab and etanercept, are unclear and possibly harmful.

CN 102973593 discloses the use of dextran sulfate in preparing a medicament for treating hepatic fibrosis.

Hence, there is still a need for a treatment that can improve liver functions in subjects suffering from a fatty liver disease.

### SUMMARY

It is a general objective to improve liver function of subjects suffering from a fatty liver disease.

It is a particular objective to provide a treatment for fatty liver diseases.

These and other objectives are met by embodiments as disclosed herein.

The present invention is defined in the independent claims. Further embodiments of the invention are defined by the dependent claims.

An aspect of the invention relates to dextran sulfate, or a pharmaceutically acceptable salt thereof, having an average molecular weight equal to or below 10 000 Da for use in improving liver function in a subject suffering from a non-alcoholic fatty liver disease (NAFLD). The dextran sulfate, or the pharmaceutically acceptable salt thereof, is formulated for intravenous or subcutaneous administration to the subject.

Another aspect of the invention relates to dextran sulfate, or a pharmaceutically acceptable salt thereof, having an average molecular weight equal to or below 10 000 Da for use in treatment of a NAFLD in a subject. The dextran sulfate, or the pharmaceutically acceptable salt thereof, is formulated for intravenous or subcutaneous administration to the subject.

Dextran sulfate, or a pharmaceutically acceptable salt thereof, according to the embodiments causes an improvement in liver function in subjects as assessed using the biomarker bilirubin. Accordingly, dextran sulfate, or the pharmaceutically acceptable salt thereof, according to the embodiments can be used in the treatment of fatty liver diseases including NAFLD.

### BRIEF DESCRIPTION OF THE DRAWINGS

The embodiments, together with further objects and advantages thereof, may best be understood by making reference to the following description taken together with the accompanying drawings, in which:

FIG. 1 illustrates total bilirubin levels in the serum of patients after dextran sulfate treatment.

FIG. 2 illustrates log ratio data relating to the cellular and molecular responses of human 3BT (B cells+peripheral blood mononuclear cells), CASM3C (coronary artery smooth muscle cells) and HDF3CGF (dermal fibroblasts) systems to 150, 400, 1300 and 4000 nM of dextran sulfate. The response readouts are indicated along the x-axis and any that are deemed significant are labelled on the graph. The control response is represented by the grey area.

FIG. 3 is a summary of dextran sulfate-induced responses across all 12 BioMAP® Diversity Plus human cell systems.

FIG. 4 dextran sulfate was referenced against Pirfenidone, an anti-fibrotic compound approved for the treatment of idiopathic pulmonary fibrosis. Dextran sulfate and Pirfenidone have 5 common activities (annotated) and 34 differential activities that were outside the normal range (grey).

FIG. 5 shows a summary of differentially regulated genes in human Schwann cell cultures over 48 hours in the absence (A) and presence of dextran sulfate (B). Full box denotes the upregulated genes, dashed box indicates the downregulated genes. The full arrows indicate activation of the inflammatory response in the Schwann cell cultures under normal culture conditions (A); the dashed arrows indicate inhibition of the expression of inflammatory genes by dextran sulfate (B). TGFβ regulates the expression of a large number of molecules. The molecular interactions allow the prediction of the downstream effects of TGFβ. In human Schwann cell cultures (C), TGFβ activation elicits fibrosis and the activation of immune cells, while dextran sulfate treatment inhibits these changes, with the exception of cell movement.

### DETAILED DESCRIPTION

The present embodiments generally relate to fatty liver diseases, and treatment thereof using dextran sulfate, or a pharmaceutically acceptable salt thereof.

Dextran sulfate of the present invention was administered to human patients in an ongoing study for treatment of a neurological disease. It was then highly surprising that dextran sulfate did not only have beneficial effects relating to the treatment of the neurological disease but also significantly improved the liver function of the patients. In fact, weekly administration of dextran sulfate of the embodiments significantly decreased total bilirubin in the patients as compared to total bilirubin levels prior to dextran sulfate administration.

Total bilirubin, i.e., free bilirubin plus conjugated bilirubin, is routinely used as a biomarker of liver function. A medical condition that negatively affects the function of the liver can cause bilirubin build up in the blood since the liver loses its ability to remove and process bilirubin from the bloodstream.

Fatty liver disease (FLD), also known as hepatic steatosis, is a condition where excess fat builds up in the liver. There are two types of fatty liver disease: non-alcoholic fatty liver disease (NAFLD) and alcoholic liver disease (ALD).

NAFLD, also known as metabolic (dysfunction) associated fatty liver disease (MAFLD), is excessive fat build-up in the liver. NAFLD is the most common liver disorder worldwide and is present in approximately 25% of the world's population. NAFLD is the leading cause of chronic liver disease and the second most common reason for liver transplantation in the US and Europe as of 2017. Obesity and type 2 diabetes are strong risk factors for NAFLD. Other risks include being overweight, metabolic syndrome, a diet high in fructose, and older age.

There are two types of NAFLD; non-alcoholic fatty liver (NAFL) and non-alcoholic steatohepatitis (NASH), with the latter also including liver inflammation. These diseases begin with fatty accumulation in the liver (hepatic steatosis). A liver can remain fatty without disturbing liver function as in NAFL, but by various mechanisms and possible insults to the liver, it may also progress into NASH, a state in which steatosis is combined with inflammation and often fibrosis (steatohepatitis). NASH can then lead to further complications such as cirrhosis and hepatocellular carcinoma.

ALD, also called alcohol-related liver disease (ARLD), is a term that encompasses the liver manifestations of alcohol overconsumption, including fatty liver, alcoholic hepatitis, and chronic hepatitis with liver fibrosis or cirrhosis. It is the major cause of liver disease in Western countries. More than 90% of all heavy drinkers develop fatty liver whilst about 25% develop the more severe alcoholic hepatitis, and 15% cirrhosis.

Experimental data as presented herein shows that dextran sulfate, or a pharmaceutically acceptable salt thereof, according to the embodiments is capable of improving liver function as seen in a significant reduction in total bilirubin. Such an improvement of liver function is beneficial to subjects suffering from a liver disease, including FLD and in particular NAFLD, such as NAFL or NASH.

In addition, dextran sulfate according to the embodiments was capable of modulating inflammatory and tissue remodeling pathways in human cells. The results as presented herein indicate that dextran sulfate modulated key innate and adaptive responses relating to immune activation, inflammation resolution and tissue remodeling ultimately leading to improved healing and function remodeling of diseased and damaged human tissues.

Hence, an aspect of the invention relates to dextran sulfate, or a pharmaceutically acceptable salt thereof, having an average molecular weight equal to or below 10 000 Da for use in improving liver function in a subject suffering from a NAFLD. The dextran sulfate, or the pharmaceutically acceptable salt thereof, is formulated for intravenous or subcutaneous administration to the subject.

A related aspect of the invention defines dextran sulfate, or a pharmaceutically acceptable salt thereof, having an average molecular weight equal to or below 10 000 Da for use in treatment of a NAFLD in a subject. The dextran sulfate, or the pharmaceutically acceptable salt thereof, is formulated for intravenous or subcutaneous administration to the subject.

The present invention also relates to use of dextran sulfate, or a pharmaceutically acceptable salt thereof, having an average molecular weight equal to or below 10 000 Da in the manufacture of a medicament for improving liver function in a subject suffering from a NAFLD and in the manufacture of a medicament for treatment of a NAFLD in a subject. The medicament is formulated for intravenous or subcutaneous administration to the subject.

A further aspect of the invention relates to a method for improving liver function in a subject. The method comprising administering dextran sulfate, or a pharmaceutically acceptable salt thereof, having an average molecular weight equal to or below 10 000 Da by intravenous or subcutaneous administration to a subject suffering from a NAFLD to improve the liver function of the subject. Yet another aspect of the invention relates to a method for treating a NAFLD. The method comprises administering dextran sulfate, or a pharmaceutically acceptable salt thereof, having an average molecular weight equal to or below 10 000 Da by intravenous or subcutaneous administration to a subject suffering from a NAFLD to treat NAFLD in the subject.

In an embodiment, the NAFLD is non-alcoholic fatty liver (NAFL).

In another embodiment, the NAFLD is non-alcoholic steatohepatitis (NASH).

The dextran sulfate, or the pharmaceutically acceptable salt thereof, is formulated for systemic administration to the subject.

The dextran sulfate, or the pharmaceutically acceptable salt thereof, is formulated for intravenous (i.v.) or subcutaneous (s.c.) administration to the subject. Accordingly, i.v. and s.c. administration are preferred examples of systemic administration of dextran sulfate, or the pharmaceutically acceptable salt thereof. In a particular embodiment, dextran sulfate, or the pharmaceutically acceptable salt thereof, is formulated for s.c. administration to the subject.

In an embodiment, dextran sulfate, or the pharmaceutically acceptable salt thereof, is formulated as an aqueous injection solution, preferably as an aqueous i.v. or s.c. injection solution. Thus, dextran sulfate, or the pharmaceutically acceptable salt thereof, of the embodiments is preferably formulated as an aqueous injection solution with a selected solvent or excipient. The solvent is advantageously an aqueous solvent and in particular a buffer solution. A non-limiting example of such a buffer solution is a citric acid buffer, such as citric acid monohydrate (CAM) buffer, or a phosphate buffer. For instance, dextran sulfate of the embodiments can be dissolved in saline, such as 0.9% NaCl saline, and then optionally buffered with 75 mM CAM and adjusting the pH to about 5.9 using sodium hydroxide. Also non-buffered solutions are possible, including aqueous injection solutions, such as saline, i.e., NaCl (aq). Furthermore, other buffer systems than CAM and phosphate buffers could be used if a buffered solution is desired.

The active compound, dextran sulfate, is then formulated with a suitable excipient, solvent or carrier that is selected based on the particular administration route.

Carrier refers to a substance that serves as a vehicle for improving the efficiency of delivery and/or the effectiveness of dextran sulfate, or the pharmaceutically acceptable salt thereof.

Excipient refers to a pharmacologically inactive substance that is formulated in combination with dextran sulfate, or the pharmaceutically acceptable salt thereof, and includes, for instance, bulking agents, fillers, diluents and products used for facilitating drug absorption or solubility or for other pharmacokinetic considerations.

Pharmaceutically acceptable salt of dextran sulfate refers to a salt of dextran sulfate having the effects as disclosed herein and not being deleterious to the recipient thereof at the administered dose(s).

The dextran sulfate is a so-called low molecular weight dextran sulfate.

In the following, reference to (average) molecular weight and sulfur content of dextran sulfate applies also to any pharmaceutically acceptable salt of dextran sulfate. Hence, the pharmaceutically acceptable salt of dextran sulfate preferably has the average molecular weight and sulfur content as discussed in the following embodiments.

Dextran sulfate is a sulfated polysaccharide and in particular a sulfated glucan, i.e., a polysaccharide made of many glucose molecules. Average molecular weight as defined herein indicates that individual sulfated polysaccharides may have a molecular weight different from this average molecular weight but that the average molecular weight represents the mean molecular weight of the sulfated polysaccharides. This further implies that there will be a natural distribution of molecular weights around this average molecular weight for a dextran sulfate sample.

Average molecular weight (M_{w}) of dextran sulfate is typically determined using indirect methods, such as gel exclusion/penetration chromatography, light scattering or viscosity. Determination of average molecular weight using such indirect methods will depend on a number of factors, including choice of column and eluent, flow rate, calibration procedures, etc.

Average molecular weight (M_{w}): ΣMᵢ²Nᵢ/ΣMᵢNᵢ, typical for methods sensitive to molecular size rather than numerical value, e.g., light scattering and size exclusion chromatography (SEC) methods. If a normal distribution is assumed, then a same weight on each side of M_{w} i.e., the total weight of dextran sulfate molecules in the sample having a molecular weight below M_{w} is equal to the total weight of dextran sulfate molecules in the sample having a molecular weight above M_{w}.

The dextran sulfate, or the pharmaceutically acceptable salt thereof, has an average molecular weight equal to or below 10 000 Da.

Dextran sulfate of an average molecular weight exceeding 10 000 Da generally has a lower effect vs. toxicity profile as compared to dextran sulfate having a lower average molecular weight. This means that the maximum dose of dextran sulfate that can be safely administered to a subject is lower for larger dextran sulfate molecules (>10 000 Da) as compared to dextran sulfate molecules having an average molecular weight within the preferred range. As a consequence, such larger dextran sulfate molecules are less appropriate in clinical uses when the dextran sulfate is to be administered to subjects in vivo.

In an embodiment, dextran sulfate, or the pharmaceutically acceptable salt thereof, has an average molecular weight within a range of from 2 000 to 10 000 Da. In another embodiment, the average molecular weight is within a range of from 2 500 to 10 000 Da. In a particular preferred embodiment, the average molecular weight is within a range of from 3 000 to 10 000 Da.

In an optional, but preferred embodiment, less than 40% of the dextran sulfate molecules have a molecular weight below 3 000 Da, preferably less than 35%, such as less than 30% or less than 25% of the dextran sulfate molecules have a molecular weight below 3 000 Da. In addition, or alternatively, less than 20% of the dextran sulfate molecules have a molecular weight above 10 000 Da, preferably less than 15%, such as less than 10% or less than 5% of the dextran sulfate molecules have a molecular weight above 10 000 Da. Thus, in a particular embodiment, the dextran sulfate has a substantially narrow molecular weight distribution around the average molecular weight.

In a particular embodiment, the average molecular weight of dextran sulfate, or the pharmaceutically acceptable salt thereof, is within a range of from 3 500 to 9 500 Da, such as within a range of from 3 500 to 8 000 Da.

In another particular embodiment, the average molecular weight of dextran sulfate, or the pharmaceutically acceptable salt thereof, is within a range of from 4 500 to 7 500 Da.

In a further particular embodiment, the average molecular weight of dextran sulfate, or the pharmaceutically acceptable salt thereof, is within a range of from 4 500 to 5 500 Da.

Thus, in a currently preferred embodiment the average molecular weight of dextran sulfate, or the pharmaceutically acceptable salt thereof, is preferably approximately 5 000 Da or at least substantially close to 5 000 Da, such as 5 000±500 Da, for instance 5 000±400 Da, preferably 5 000±300 Da or 5 000±200 Da, such as 5 000±100 Da. Hence, in an embodiment, the average molecular weight of dextran sulfate, or the pharmaceutically acceptable salt thereof, is 4.5 kDa, 4.6 kDa, 4.7 kDa, 4.8 kDa, 4.9 kDa, 5.0 kDa, 5.1 kDa, 5.2 kDa, 5.3 kDa, 5.4 kDa or 5.5 kDa.

In a particular embodiment, the average molecular weight of dextran sulfate, or the pharmaceutically salt thereof as presented above is average M_{w} and preferably determined by gel exclusion/penetration chromatography, size exclusion chromatography, light scattering or viscosity-based methods.

Dextran sulfate is a polyanionic derivate of dextran and contains sulfur. The average sulfur content for dextran sulfate of the embodiments is preferably from 15 to 20% and more preferably approximately 17%, generally corresponding to about or at least two sulfate groups per glucosyl residue. In a particular embodiment, the sulfur content of dextran sulfate is preferably equal to or at least close to the maximum possible degree of sulfur content of the corresponding dextran molecules.

In a particular embodiment, dextran sulfate of the embodiments has a number average molecular weight (Mₙ) as measured by nuclear magnetic resonance (NMR) spectroscopy within a range of from 1850 to 3500 Da.

Number average molecular weight (Mₙ):

$$\frac{\sum {M}_{i} {N}_{i}}{\sum {N}_{i}} ,$$

typically derived by end group assays, e.g., NMR spectroscopy or chromatography. If a normal distribution is assumed, then a same number of dextran sulfate molecules can be found on each side of Mₙ, i.e., the number of dextran sulfate molecules in the sample having a molecular weight below Mₙ is equal to the number of dextran sulfate molecules in the sample having a molecular weight above Mₙ.

In a preferred embodiment, dextran sulfate of the embodiments has a Mₙ as measured by NMR spectroscopy within a range of from 1850 to 2500 Da, preferably within a range of from 1850 to 2300 Da, and more preferably within a range of from 1850 to 2000 Da.

In a particular embodiment, dextran sulfate of the embodiments has an average sulfate number per glucose unit within a range of from 2.5 to 3.0, preferably within a range of from 2.5 to 2.8, and more preferably within a range of from 2.6 to 2.7.

In a particular embodiment, dextran sulfate of the embodiments has an average number of glucose units within a range of from 4.0 to 6.0, preferably within a range of from 4.5 to 5.5, and more preferably within a range of from 5.0 to 5.2, such as about 5.1.

In another particular embodiment, dextran sulfate of the embodiments has on average 5.1 glucose units and an average sulfate number per glucose unit of 2.6 to 2.7, typically resulting in a number average molecular weight (Mₙ) as measured by NMR spectroscopy within a range of from 1850 to 2000 Da.

A dextran sulfate, or pharmaceutically salt thereof, that can be used according to the embodiments is described in WO 2016/076780.

The dextran sulfate according to the embodiments can be provided as a pharmaceutically acceptable salt of dextran sulfate. Such pharmaceutically acceptable salts include e.g., a sodium or potassium salt of dextran sulfate. In a particular embodiment, the pharmaceutically acceptable salt is a sodium salt of dextran sulfate.

In a particular embodiment, the sodium salt of dextran sulfate, including Na⁺ counter ions, has a Mₙ as measured by NMR spectroscopy within a range of from 2000 to 2500 Da, preferably within a range of 2100 and 2300 Da.

In an embodiment, an effective amount of dextran sulfate, or the pharmaceutically acceptable salt thereof, is administered to the subject. Effective amount as used herein relates to a therapeutically effective amount of dextran sulfate, or the pharmaceutically acceptable salt thereof, capable of causing a medical effect when administered to the subject that is related to an improvement of the liver function and status of the subject. Such a therapeutically effective amount is preferably an amount of dextran sulfate, or the pharmaceutically acceptable salt thereof, capable of inducing a change in at least one biomarker associated with liver function, such as bilirubin. The therapeutically effective amount of dextran sulfate, or the pharmaceutically salt thereof, can be determined by the physician and may, optionally, be selected based on at least one among the sex of the subject, the weight of the subject, the age of the subject, the type of NAFLD and the severity of the NAFLD.

Suitable dose ranges for the dextran sulfate, or the pharmaceutically acceptable salt thereof, of the embodiments may vary according to the size and weight of the subject, the condition for which the subject is treated, and other considerations. In particular for human subjects, a possible dosage range could be from 1 μg/kg to 150 mg/kg of body weight, preferably from 10 μg/kg to 100 mg/kg of body weight.

In preferred embodiments, dextran sulfate, or the pharmaceutically acceptable salt thereof, is formulated to be administered at a dosage in a range from 0.05 to 50 mg/kg of body weight of the subject, preferably from 0.05 or 0.1 to 40 mg/kg of body weight of the subject, and more preferably from 0.05 or 0.1 to 30 mg/kg, or 0.1 to 25 mg/kg or from 0.1 to 15 mg/kg or 0.1 to 10 mg/kg body weight of the subject. A currently preferred dosage of dextran sulfate, or the pharmaceutically acceptable salt thereof, is from 0.5 to 5 mg/kg body weight of the subject.

Administration of dextran sulfate, or the pharmaceutically acceptable salt thereof, does not necessarily have to be limited to treatment of a NAFLD but could alternatively, or in addition, be used for prophylaxis. In other words, dextran sulfate of the embodiments could be administered to a subject having an increased risk of developing a NAFLD.

Treatment of a NAFLD also encompasses inhibition of a NAFLD. Inhibit of a NAFLD as used herein implies that dextran sulfate, or the pharmaceutically acceptable salt thereof, reduces the symptoms and effects of the condition even though a 100% treatment or cure does not necessarily occur. For instance, inhibition of a NAFLD may involve an improvement in liver function, such as seen in a decrease in the circulating levels of total bilirubin.

Dextran sulfate, or the pharmaceutically acceptable salt thereof, of the embodiments can be administered at a single administration occasion, such as in the form of a single injection or bolus injection. This bolus dose can be injected quite quickly to the subject but is advantageously infused over time so that the dextran sulfate solution is infused over a few minutes of time to the subject, such as during 5 to 10 minutes or more.

Alternatively, dextran sulfate, or the pharmaceutically acceptable salt thereof, of the embodiments can be administered at multiple, i.e., at least two, occasions during a treatment period. Thus, dextran sulfate of the embodiments could be administered once or at multiple times per day, once or at multiple times per week, once or at multiple times per month as illustrative examples.

In a particular embodiment, dextran sulfate, or the pharmaceutically acceptable salt thereof, is formulated for administration at 1-14 times, preferably 1-7 times, a week for one or multiple consecutive weeks, such as at least 2-5 consecutive weeks. In a particular embodiment, dextran sulfate, or the pharmaceutically acceptable salt thereof, is formulated for administration once or twice a day for multiple days, such as multiple consecutive days, e.g., 2-14 days.

It is also possible combine a bolus injection of dextran sulfate, or the pharmaceutically acceptable salt thereof, with one or more additional administrations of dextran sulfate, or the pharmaceutically acceptable salt thereof.

In an embodiment, the subject is a mammalian subject, preferably a primate, and more preferably a human subject. Although the embodiments are in particular directed towards treatment of fatty liver diseases, including NAFLD, in human subjects, the embodiments may also, or alternatively, be used in veterinary applications. Non-limiting example of animal subjects include non-human primate, cat, dog, pig, horse, mouse, rat, goat, guinea pig, sheep and cow.

### EXAMPLES

### Example 1

Using a longitudinal design, this study was aimed to determine the changes of selected serum metabolites in patients before dextran sulfate administration and after different times following the beginning of the treatment. Changes in the measured metabolites indicated the biochemical response of the patient to dextran sulfate that is underpinning potential disease modification and the mechanisms of action of the drug in this patient population.

### Materials and Methods

Dextran sulfate (Tikomed A B, Viken, Sweden, WO 2016/076780) was administered at 2 mg/kg by daily subcutaneous injection once a week for ten weeks to human patients suffering from a neurological disease.

Peripheral venous blood samples were collected from patients before (week 0) and after dextran sulfate administration (week 5 and week 10) after at least 15 minutes of complete rest, using the standard tourniquet procedure, from the antecubital vein into a single VACUETTE® polypropylene tube containing serum separator and clot activator (Greiner-Bio One GmbH, Kremsmunster, Austria). After 30 mins at room temperature (20-25° C.), blood withdrawals were centrifuged at 1,890×g for 10 min to get serum aliquots.

A serum aliquot of about 300 μl was light-protected and then processed to extract fat-soluble metabolites using a method described in detail (Lazzarino et al., Single-step preparation of selected biological fluids for the high performance liquid chromatographic analysis of fat-soluble vitamins and antioxidants, J Chromatogr A. 2017; 1527: 43-52). Briefly, samples were supplemented with 1 ml of HPLC-grade acetonitrile, vigorously vortexed for 60 sec and incubated at 37° C. for 1 h in a water bath under agitation to allow the full extraction of lipid soluble compounds. Samples were then centrifuged at 20,690×g for 15 min at 4° C. to precipitate proteins and the clear supernatants were saved at −80° C. until the HPLC analysis of fat-soluble metabolites.

### Results

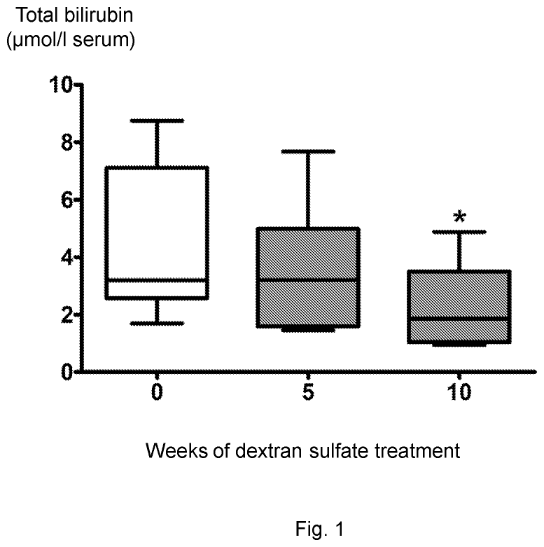

Dextran sulfate administration produced a significant time-dependent decrease in the circulating levels of total bilirubin (total bilirubin=free bilirubin+conjugated bilirubin) reaching significant differences at week 10 of treatment as compared to week 0 of treatment (*p<0.05) as shown in FIG. 1.

The results show that dextran sulfate significantly improved liver function in the patients and thereby is useful in treatment of fatty liver diseases including NAFLD.

### Example 2

Inflammation precedes fibrosis in fibroproliferative conditions. This example investigated the effects of dextran sulfate in modulating inflammatory and tissue remodeling pathways in human cells. Protein and gene expression data from relevant cultured human cells suggested that, through reprogramming immune activation, inflammation resolution and tissue remodeling, dextran sulfate modulated key innate and adaptive responses leading to improved healing and functional remodeling of diseased and damaged human tissues.

### Materials and Methods

Dextran Sulfate

Dextran sulfate (Tikomed A B, Viken, Sweden, WO 2016/076780) was provided as a stock concentration of 20 mg/ml and was kept in a temperature monitored refrigerator. Immediately prior to use dextran sulfate aliquots were diluted to the appropriate concentration in sterile saline.

Phenotypic Profiling Using BioMAP® Systems

Twelve primary human cell and co-cultures assays were used to assess the effects of dextran sulfate on clinically-relevant protein biomarkers of inflammation, cell growth and fibrosis as part of the quality controlled BioMAP® Diversity PLUS, commercially available service (Eurofins DiscoverX Corporation, Freemont, CA, USA; for full details https://www.discoverx.com). Dextran sulfate was tested in these assays at four concentrations: 4000 nMw 1300 nMw 400 nM and 150 nM. Human primary cells employed in the BioMAP® systems were used at passage 4 or earlier, derived from multiple donors (n=2 to 6), commercially purchased and handled according to the recommendations of the manufacturers. Human blood derived CD14+ monocytes were differentiated into macrophages in vitro before being added to the IMphg system (Eurofins DiscoverX Corporation).

The cell types and stimuli used in each assay system were as follows: 3C system [Human umbilical vein endothelial cells (HUVEC)+(IL-1β, TNFα and IFNγ)], 4H system [HUVEC+(IL-4 and histamine)], LPS system [Peripheral blood mononuclear cells (PBMC) and HUVEC+LPS (TLR4 ligand)], SAg system [PBMC and HUVEC+TCR ligands], BT system [CD19+ B cells and PBMC+(α-IgM and TCR ligands)], BF4T system [bronchial epithelial cells (BEC) and human neonatal dermal fibroblasts (HDFn)+(TNFα and IL-4)], BE3C system [BEC+(IL-1β, TNFα and IFNγ)], CASM3C system [coronary artery smooth muscle cells (CASMC)+(IL-1β, TNFα and IFNγ)], HDF3CGF system [HDFn+(IL-1β, TNFα, IFNγ, EGF, bFGF and PDGF-BB)], KF3CT system [keratinocytes and HDFn+(IL-1β, TNFα, IFNγ and TGFβ)], MyoF system [differentiated lung myofibroblasts+(TNFα and TGFβ)] and IMphg system [HUVEC and M1 macrophages+Zymosan (TLR2 ligand)].

Assays were derived from either single cell types or co-culture systems. Adherent cell types were cultured in 96 or 384-well plates until confluence, followed by the addition of PBMC (SAg and LPS systems). The BT system consisted of CD19+ B cells co-cultured with PBMC and stimulated with a BCR activator and low levels of TCR stimulation. Test agents prepared in either DMSO (small molecules; final concentration 0.1%) or PBS (biologics) were added at the indicated concentrations 1 h before stimulation, and cells remained in culture for 24 h or as otherwise indicated (48 h, MyoF system; 72 h, BT system (soluble readouts); 168 h, BT system (secreted IgG)). Each assay plate contained drug controls (e.g., legacy control test agent colchicine at 1.1 μM), negative controls (e.g., non-stimulated conditions) and vehicle controls (e.g., 0.1% DMSO) appropriate for each system. Direct ELISA was used to measure biomarker levels of cell-associated and cell membrane targets. Soluble factors from supernatants were quantified using either HTRF® detection, bead-based multiplex immunoassay or capture ELISA. Overt adverse effects of dextran sulfate on cell proliferation and viability (cytotoxicity) were detected by sulforhodamine B (SRB) staining for adherent cells, and alamarBlue® reduction for cells in suspension. For proliferation assays, individual cell types were cultured at subconfluence and measured at time points optimized for each system (48 h: 3C and CASM3C systems; 72 h: BT and HDF3CGF systems; 96 h: SAg system). Cytotoxicity for adherent cells was measured by SRB (24 h: 3C, 4H, LPS, SAg, BF4T, BE3C, CASM3C, HDF3CGF, KF3CT, and IMphg systems; 48 h: MyoF system), and by alamarBlue® staining for cells in suspension (24 h: SAg system; 42 h: BT system) at the time points indicated.

BioMAP® Data Analysis

Biomarker measurements in dextran sulfate-treated samples were divided by the average of the control samples (at least 6 vehicle controls from the same plate) to generate a ratio that was then log₁₀ transformed. Significance prediction envelopes were calculated using historical vehicle control data at a 95% confidence interval. Biomarker activities were annotated when 2 or more consecutive concentrations change in the same direction relative to vehicle controls were outside of the significance envelope and had at least one concentration with an effect size >20% (log₁₀ ratio>0.1). Biomarker key activities were described as modulated if these activities increase in some systems but decrease in others. Cytotoxic conditions were noted when total protein levels decreased by more than 50% (log₁₀ ratio of SRB or alamarBlue® levels <−0.3) and were indicated by a thin black arrow above the X-axis. A compound was considered to have broad cytotoxicity when cytotoxicity was detected in 3 or more systems. Concentrations of test agents with detectable broad cytotoxicity were excluded from biomarker activity annotation and downstream benchmarking, similarity search and cluster analysis. Antiproliferative effects were defined by an SRB or alamarBlue® log₁₀ ratio value <−0.1 from cells plated at a lower density and were indicated by grey arrows above the X-axis. Cytotoxicity and antiproliferative arrows only require one concentration to meet the indicated threshold for profile annotation.

Gene Expression Studies

Human Schwann cells (ATCC CRL-2884™) were cultured in 25 cm² culture flasks coated with poly-d-lysine and laminin) in high-glucose DMEM with 10% fetal bovine serum (FBS) (n=8) and incubated at 37° C./5% CO₂. Twenty-four house after seeding (Day 0) cells were treated once with either dextran sulfate (0.01 mg/ml in DMEM) or culture media. After 48 hours with treatment, the media were removed and cells were harvested in 2.5 ml Trizol:Water (4:1) solution containing 0.5 ml of chloroform at room temperature (RT). The flasks were inspected under the microscope to ensure full removal of cells. The collected lysates were stored at −80° C.

RNA Extraction for Gene Expression Studies

Lysates were left to equilibrate for 5 minutes at room temperature to permit the complete dissociation of nucleoprotein complexes. 1 ml lysate was removed from each sample and 200 μL of chloroform was added to each and the tube was shaken vigorously. Samples were stored at room temperature for 2-3 minutes and subsequently centrifuged at 12,000×g/15 min at 4° C. The mixture separated into 3 layers: a lower red phenol-chloroform phase, an interphase and a colorless upper aqueous phase. The top % of the aqueous phase (containing the RNA) was transferred to a new clean Eppendorf tube. The RNA was precipitated from the aqueous phase by adding an equal amount of 100% ethanol. The precipitated RNA was fixed onto a Spin Cartridge, washed twice and dried. The RNA was eluted in 50 μL warm RNase-Free Water. The amount and quality of the purified RNA was measured by Nanodrop. The RNA was stored at −80° C. before transfer to Source Bioscience for Gene Array analysis. The additional QC from the array service provider (Source Bioscience) indicated that the RNA was high quality (no degradation) and the amounts were well within the parameters of the Low Input RNA Microarray from Agilent.

Analysis of Gene Expression Data

The background corrected expression data from each sample were downloaded into a separate file. The background corrected signal was log₂ transformed for all samples for statistical analysis. To reduce the false discovery rate in the samples, the signals that were below ‘expression level’ were removed. The ‘below expression’ level was set at 5 for the _{log2} transformed expression values. (The expression value of the MAPT gene, which is not normally expressed in Schwann cells, was <5. However, the expression of usual ‘control’ gene ACTB (A_32_P137939; ACTB) was comfortably above 5).

Statistical Analysis of the Gene Expression Data

MetaboAnalyst (Chong and Xia, MetaboAnalystR: an R package for flexible and reproducible analysis of metabolomics data, Bioinformatics 34(24): 4313-4314 (2018)) was used to analyze the gene expression profiles. Based on the expression pattern of the control probes on each array it was decided to carry out Median Centring for all arrays before analysis to reduce the variability of the results.

Additionally, a preliminary analysis was carried out to screen out genes that were not differentially expressed between any combinations of the three datasets. Simple, non-stringent ANOVA (p<0.05) was carried out to look for patterns of expression. Probes with no changes across the three datasets were eliminated. The remaining probe sets were analyzed for fold change and significance using Volcano plots (Qlucore, Lund, Sweden). More than 20% change in the expression of a probe (FC≥1.2 or FC≤0.84) was regarded as significant in the first instance to allow the detection of expression patterns.

The comparison of Day 0 Control samples to the Day 2 Control samples allowed the analysis of the gene expression changes seen in the cells during normal culture conditions. The effect of dextran sulfate was regarded as the difference between the Day 2 Control versus the Day 2 dextran sulfate treated samples. The genes differentially regulated and the fold change values from this analysis were uploaded to the Ingenuity Pathway Analysis (IPA; Qiagen Ltd., Manchester, UK) software. Following a core analysis of the differential expression profiles, comparison analyses were performed to provide a complete profile of the dextran sulfate effects. The TGFβ regulated mechanistic network of interest was built from genes known to be associated with fibrosis and scarring.

### Results

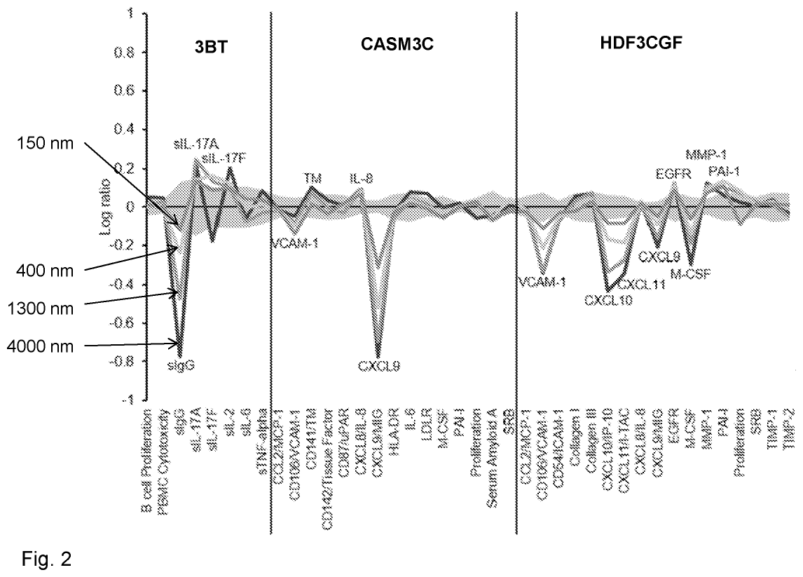

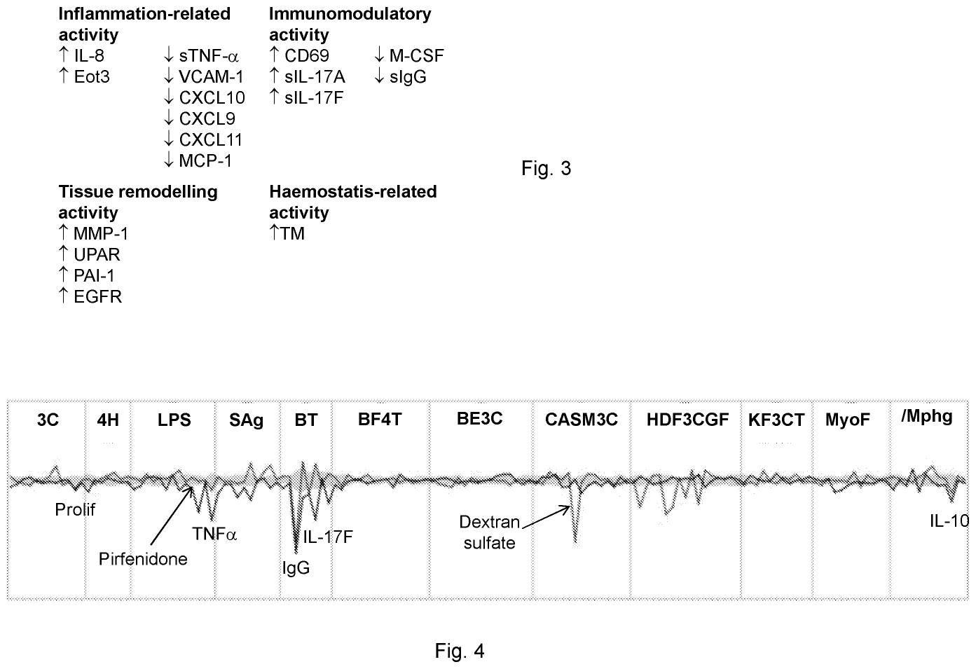

Dextran sulfate modulates inflammatory and wound healing responses in cultured human cells BioMAP® has been developed as a method to assess efficacy, safety and the mechanism of action of drugs in multiple human cell types stimulated with inflammatory challenges. The BioMAP® cellular responses that were most affected by dextran sulfate related to T cell-dependent activation of B cells (BT system), the Th1 inflammatory environment for arterial smooth cells (CASM3C system) and the tissue remodeling responses of human dermal fibroblasts (HDF3CGF system) (FIG. 2). Importantly, there was no cytotoxicity evident with dextran sulfate in any of the human culture systems evaluated. Overall analysis of the responses of the 12 model systems included in the assay showed that dextran sulfate modulated inflammatory and immunoregulatory cytokines and molecules, tissue remodeling pathways (including activation of matrix degrading enzymes) and hemostasis activity (FIG. 3). These effects were benchmarked against that of pirfenidone, and dextran sulfate induced a unique anti-inflammatory profile, by targeting different inflammatory regulators after challenge in this broad range of human cell systems (FIG. 4).

Dextran Sulfate Modulates Expression of Inflammatory and Fibrogenic Genes in Cultured Human Cells

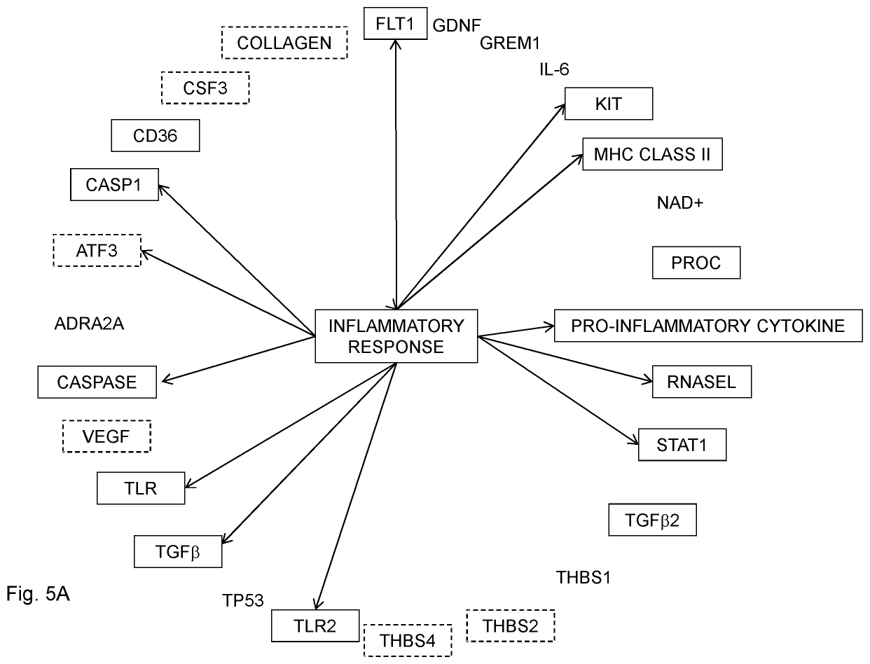

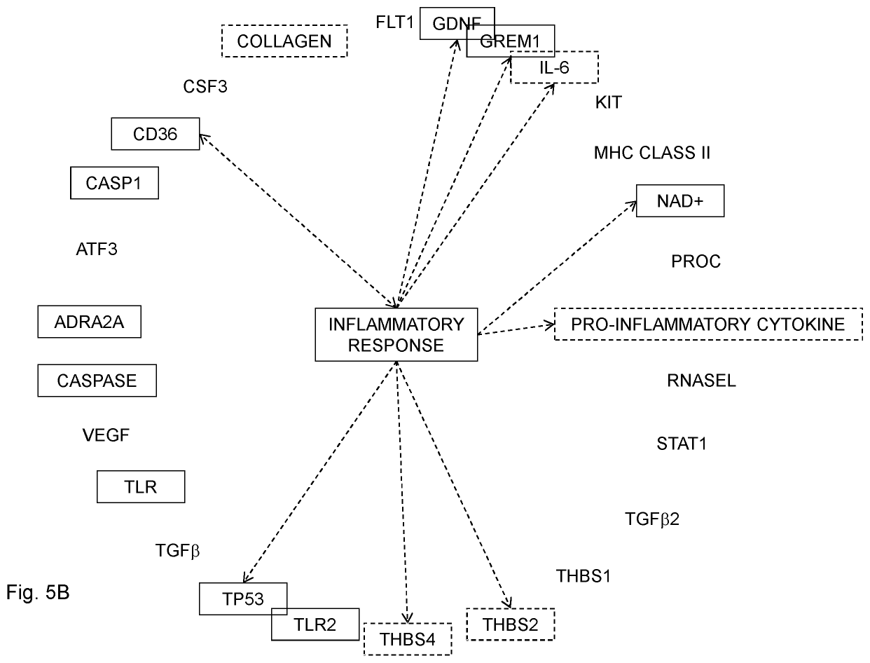

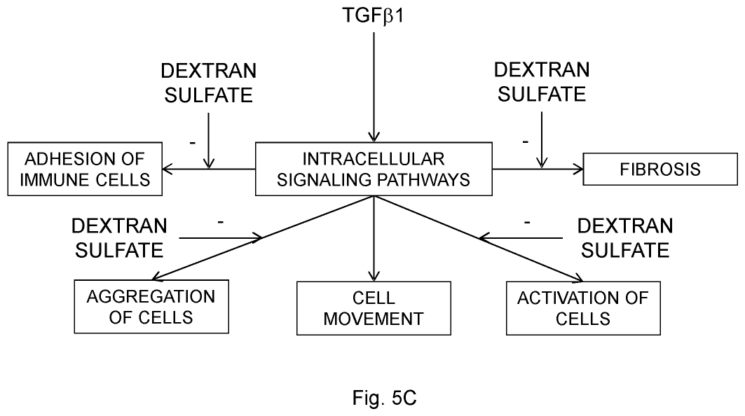

To confirm and further understand the mechanisms of dextran sulfate, and to underline the broad relevance of dextran sulfate actions, the drug impact upon gene expression in human Schwann cells was analyzed; these glia are widely used to evaluate the inflammatory and fibrotic responses seen in many degenerative disorders. Gene expression analysis demonstrated that, when placed in culture, human Schwann cells produce a robust inflammatory expression profile resulting in activation of the inflammatory response (FIG. 5A) and activation of the TGFβ signaling pathway (FIG. 5C). Similar to the BioMAP© data, molecular network analysis showed that dextran sulfate had a complex effect on the inflammatory genes, resulting in resolution of the normal inflammatory response (FIG. 5B), including targeted modulation of key elements of the signaling cascade relating to the pro-fibrotic cytokine TGFβ. The TGFβ-regulated mechanistic molecular network responsible for fibrosis includes 165 molecules and, of these, dextran sulfate regulated the expression of 14 (8.5%; p<0.001). The changes in gene expression in the TGFβ-regulated gene network indicated that dextran sulfate attenuated fibrosis by upregulating the expression of the anti-fibrotic proteoglycan decorin and modulating matrix metallopeptidases, thereby activating tissue remodeling processes.

The mechanistic profile of dextran sulfate was explored at the protein level using the BioMAP® Diversity Plus assay, which comprised 12 different validated human primary cell culture systems that modelled inflammation and wound healing responses in different human tissue types. The data demonstrated that dextran sulfate had a distinct phenotypic profile with multi-modal actions; having effects in different cell types and following stimulation with different inflammatory mediators. In particular dextran sulfate reduced levels of 3 chemokines, CXCL9, CXCL10 and CXCL11, which are all structurally related, signal through the CXC receptor 3 (CXCR3) and are induced by interferons and TNFα. CXCR3 and its associated chemokines play an important role in recruitment and function of immune cells and have been implicated in numerous inflammatory diseases.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-15. (canceled)

16. A method for improving liver function in a subject suffering from a non-alcoholic fatty liver disease (NAFLD), the method comprising intravenous or subcutaneous administering dextran sulfate, or a pharmaceutically acceptable salt thereof, having an average molecular weight equal to or below 10,000 Da to the subject suffering from NAFLD.

17. A method for treating a non-alcoholic fatty liver disease (NAFLD) in a subject, the method comprising intravenous or subcutaneous administering dextran sulfate, or a pharmaceutically acceptable salt thereof, having an average molecular weight equal to or below 10,000 Da to the subject suffering from NAFLD.

18. The method according to claim 17, wherein the NAFLD is non-alcoholic fatty liver (NAFL).

19. The method according to claim 17, wherein the NAFLD is non-alcoholic steatohepatitis (NASH).

20. The method according to claim 17, wherein administering the dextran sulfate, or the pharmaceutically acceptable salt thereof, comprises subcutaneous administering the dextran sulfate, or the pharmaceutically acceptable salt thereof, having an average molecular weight equal to or below 10,000 Da to the subject suffering from NAFLD.

21. The method according to claim 17, wherein the average molecular weight is within a range of from 2000 to 10,000 Da.

22. The method according to claim 21, wherein the average molecular weight is within a range of from 4500 to 7500 Da.

23. The method according to claim 17, wherein the dextran sulfate, or the pharmaceutically acceptable salt thereof, has an average sulfur content in a range of from 15 to 20%.

24. The method according to claim 17, wherein the dextran sulfate, or the pharmaceutically acceptable salt thereof, has a number average molecular weight (Mₙ) as measured by nuclear magnetic resonance (NMR) spectroscopy within a range of from 1850 to 3500 Da.

25. The method according to claim 24, wherein the dextran sulfate, or the pharmaceutically acceptable salt thereof, has a Mₙ as measured by NMR spectroscopy within a range of from 1850 to 2500 Da.

26. The method according to claim 25, wherein the dextran sulfate, or the pharmaceutically acceptable salt thereof, has a Mₙ as measured by NMR spectroscopy within a range of from 1850 to 2300 Da.

27. The method according to claim 26, wherein the dextran sulfate, or the pharmaceutically acceptable salt thereof, has a Mₙ as measured by NMR spectroscopy within a range of from 1850 to 2000 Da.

28. The method according to claim 24, wherein the dextran sulfate, or the pharmaceutically acceptable salt thereof, has an average sulfate number per glucose unit within a range of from 2.5 to 3.0.

29. The method according to claim 28, wherein the dextran sulfate, or the pharmaceutically acceptable salt thereof, has an average sulfate number per glucose unit within a range of from 2.5 to 2.8.

30. The method according to claim 29, wherein the dextran sulfate, or the pharmaceutically acceptable salt thereof, has an average sulfate number per glucose unit within a range of from 2.6 to 2.7.

31. The method according to claim 17, wherein the dextran sulfate, or the pharmaceutically acceptable salt thereof, has on average 5.1 glucose units and an average sulfate number per glucose unit of 2.6 to 2.7.

32. The method according to claim 17, wherein administering the dextran sulfate, or the pharmaceutically acceptable salt thereof, comprises intravenous or subcutaneous administering an aqueous injection solution comprising the dextran sulfate, or the pharmaceutically acceptable salt thereof, having an average molecular weight equal to or below 10,000 Da to the subject suffering from NAFLD.

33. The method according to claim 17, wherein the pharmaceutically acceptable salt thereof is a sodium salt of dextran sulfate.
